(19) United States
(12) Patent Application Publication
(10) Pub. No.: US 2022/0031250 A1
(43) Pub. Date: Feb. 3, 2022

(54) TOOTHBRUSH-DERIVED DIGITAL PHENOTYPES FOR UNDERSTANDING AND MODULATING BEHAVIORS AND HEALTH

(71) Applicant: THE REGENTS OF THE UNIVERSITY OF CALIFORNIA, Oakland, CA (US)

(72) Inventors: Vivek Shetty, Los Angeles (CA); Vwani Roychowdhury, Los Angeles (CA); Nosang Vincent Myung, Los Angeles (CA)

(73) Assignee: THE REGENTS OF THE UNIVERSITY OF CALIFORNIA, Oakland, CA (US)

(21) Appl. No.: 17/275,996

(22) PCT Filed: Sep. 13, 2019
(86) PCT No.: PCT/US2019/051115

§ 371 Date: Mar. 12, 2021

Related U.S. Application Data
(60) Provisional application No. 62/731,620, filed on Sep. 14, 2018.

(51) Int. Cl. A61B 5/00; G16H 40/63; A61B 5/145; A61B 5/16; A61B 5/11; A61C 17/22

(52) U.S. Cl. CPC A61B 5/6887; G16H 40/63; A61B 5/14507; A61B 5/14546; G06K 19/0723; A61B 5/165; A61B 5/11; A61C 17/22; A61B 5/0022

(57) ABSTRACT

An oral appliance includes: (1) a salivary sensor module including multiple sensors responsive to levels of different salivary analytes, and configured to generate output signals corresponding to the levels of the different salivary analytes; (2) a wireless communication module; and (3) a micro-controller connected to the salivary sensor module and the wireless communication module, and configured to derive the levels of the different salivary analytes from the output signals and direct the wireless communication module to convey the levels of the different salivary analytes to an external device.

### CROSS-REFERENCE TO RELATED APPLICATIONS

This application claims the benefit of U.S. Provisional Application No. 62/731,620, filed Sep. 14, 2018, the contents of which are incorporated herein by reference in their entirety.

### TECHNICAL FIELD

This disclosure generally relates to remote monitoring of sensor data reflective of behaviors and health states and deriving information for higher-level interpretation and diagnosis from the sensor data.

### BACKGROUND

Intraoral sensors are a relatively recent development. Comparative intraoral sensors are typically implemented in standalone devices, and are typically not linked to a data collection/analytics system. Also, sensor data are typically unstructured in the sense that the data represent raw measurements. It would be desirable to derive information for higher-level interpretation and diagnosis from the raw sensor data.

It is against this background that a need arose to develop the embodiments described herein.

### SUMMARY

In some embodiments, an oral appliance includes: (1) a salivary sensor module including multiple sensors responsive to levels of different salivary analytes, and configured to generate output signals corresponding to the levels of the different salivary analytes; (2) a wireless communication module; and (3) a micro-controller connected to the salivary sensor module and the wireless communication module, and configured to derive the levels of the different salivary analytes from the output signals and direct the wireless communication module to convey the levels of the different salivary analytes to an external device.

In additional embodiments, a computer-implemented method includes: (1) deriving structured data of a user from sensor data collected for the user; (2) collecting attributes of the user; (3) aggregating the structured data of the user and the attributes of the user with structured data of additional users and attributes of the additional users to obtain a population-level data set; (4) identifying a set of cohorts from the population-level data set; and (5) deriving a profile of the user indicative of an extent of matching of the user with the set of cohorts.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1. Schematic of an oral appliance and its architecture and features.

FIG. 2. Schematic of a power management circuit.

FIG. 3. Schematic of an oral hygiene device and its architecture and features.

FIG. 4. Schematic of data collection and transmission to a cloud server.

FIG. 5. Schematic of conversion of unstructured sensor data to structured behavioral or health data.

FIG. 6. Schematic of conversion of 9-axis measurements to dental regions being brushed using supervised approach.

FIG. 7. Schematic of conversion of 3-axis accelerometer data and 3-axis gyroscope data to Euler angles.

FIG. 8. Schematic of using transitions between dental regions to render dental region predictions more accurate.

FIG. 9. Schematic of mapping of time-series sensor data to a motionlet.

FIG. 10. Schematic of derivation of population-level models from structured behavioral or health data of individual users.

FIG. 11. Schematic of derivation of individual digital phenotypes from population-level phenotypes.

FIG. 12. Schematic of a computing device.

### DESCRIPTION

Embodiments of this disclosure involve the use of passive data measured by sensors (embedded within oral hygiene devices (e.g., toothbrushes) and oral appliances placed in the mouth) to derive precise and temporally dynamic digital phenotypes or profiles reflective of toothbrush use behaviors and oral/general health states of users in home settings. Derived through deep learning approaches, the digital phenotypes help to understand how users engage with their oral hygiene devices, obtain clinical insights on their oral/general health states through biometric data collected by the oral hygiene devices and generate computationally-driven, personalized, adaptive feedback and recommendations to shape their behaviors. Some embodiments include three main components: (a) a tooth-borne oral appliance including multiplexed sensors (e.g., biological and/or chemical sensors) with an antenna for wireless charging and communication; (b) an oral hygiene device in the form of an electric toothbrush with an integrated 9-axis inertial motion sensor and a near-field, wireless reader (charger/interrogator); and (c) a machine learning (ML)/artificial intelligence (AI) platform that converts unstructured sensor data to structured behavioral and health data and generates interpretable, multi-scale data-driven models for driving personalized feedback and behavioral interventions.

(a) Tooth-Borne Oral Appliance:

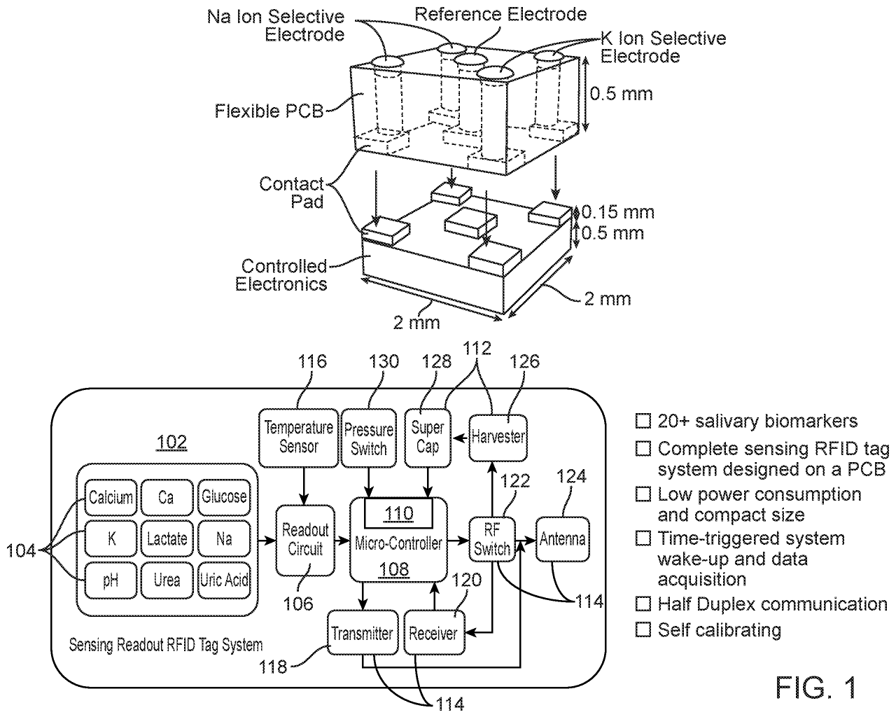

FIG. 1 shows a schematic of an oral appliance and its architecture and features of some embodiments. Implemented as a low profile, intraoral bracket bonded to a molar tooth, the oral appliance (about 3 mm by about 3 mm in area) is programmed to take snapshots of the levels of multiple (e.g., 2 or more, 5 or more, 10 or more, and up to 20 or more) salivary analytes (e.g., electrolytes/metabolites) and store data on the levels for up to about 48 hours.

To provide a high-performance system (accurate and reliable) for measurement of salivary analytes linked to health/disease states, the oral appliance is a Radio Frequency Identification (RFID)-based sensing system including a salivary sensor module 102 which includes multiple sensors 104 (e.g., biological and/or chemical sensors) including ion selective electrodes with corresponding reference electrodes, and a readout circuit 106 in the form of a potentiometric circuit. The oral appliance also includes a micro-controller 108 and an associated memory 110 to direct operation of various components of the oral appliance, a power management circuit 112, and a wireless communication module 114 in the form of a front-end RFID tag. The sensors 104 are responsive to levels of different salivary analytes, such as pH, calcium, potassium, lactate, urea, glucose, sodium, lactic acid, uric acid, creatinine, as well as other salivary electrolytes/metabolites. A diameter of the ion selective electrodes is about a few tenths of microns to a few millimeters and can be micro- or macro-fabricated on a common substrate, such as a flexible printed circuit board (PCB). The RFID-based sensing system is utilized since it can reconcile a small form-factor (can omit a battery) and consumes little power for extended periods of time. For example, the readout circuit 106 measures a potential between ion selective electrodes (working electrodes) and a reference electrode that is responsive to an analyte level, and generates an output signal corresponding to the analyte level. This measurement operation is multiplexed so as to sequentially obtain measurements across multiple sensors and across multiple salivary analytes. As shown in FIG. 1, a calibration sensor 116 in the form of a temperature sensor is included, and the calibration sensor 116 generates a calibration signal responsive to a local temperature in the mouth of a user, such that measured potentials can be adjusted or calibrated according to such calibration signal.

An output of the readout circuit 106 is fed into the micro-controller 108 to convert the output into a digital format and to derive analyte levels from measured potentials. The micro-controller 108 also manages the interfaces with the memory 110, the power management circuit 112, and the front-end RFID tag 114 to store and to communicate data. The front-end RFID tag 114 includes a transmitter module 118, a receiver module 120, an RF switch 122, and an antenna 124. Data indicative of analyte levels stored in the memory 110 is fed into the front-end RFID tag 114 through the micro-controller 108 and ultimately is transmitted through the transmitter module 118 and the antenna 124 to a near-field reader integrated within an electric toothbrush. Data also can be received from the electric toothbrush through the antenna 124 and the receiver module 120, so as to adjust operation or programming of the micro-controller 108. Additionally, RF power from the reader (during brushing) is received through the antenna 124 of the front-end RFID tag 114, and is fed into the power management circuit 112 to convert to a stable direct current (DC) voltage for powering sensing operations as well as data transmission operations.

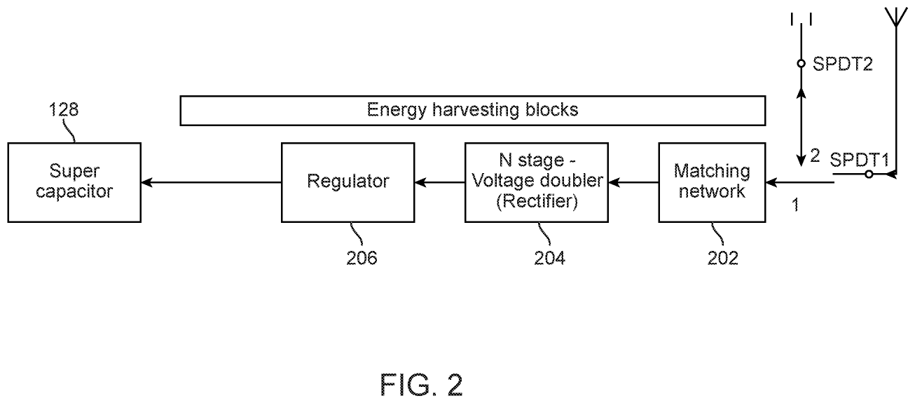

As shown in FIG. 1, the power management circuit 112 includes a harvester 126 connected to the antenna 124 through the RF switch 122, and an energy storage module 128 in the form of a super-capacitor connected to the harvester 126. The harvester 126 converts RF power to a DC voltage, which is stored in the super-capacitor 128 for powering components of the oral appliance when activated from a sleep mode to an active mode. FIG. 2 shows a schematic of the power management circuit 112 of some embodiments. As shown in FIG. 2, the harvester 126 includes a matching network 202 to receive RF power from the antenna 124, a rectifier 204, and regulator 206. The rectifier 204 converts the RF power into a DC signal that is fed to the regulator 206. An N-stage (e.g., four-stage) voltage doubler can be used as the rectifier 204 so that an output of the rectifier 204 falls within an input range of the regulator 206. The regulator 206 operates to store energy in the super-capacitor 128. A low-dropout (LDO) architecture can be used so that an output of the regulator 206 is stable with respect to any changes in an input to the regulator 206.

Referring back to FIG. 1, the oral appliance includes a pressure sensor 130, which senses chewing forces and generates a wake-up signal as an event-triggered signal. Responsive to this wake-up signal, the micro-controller 108 activates and changes a state of various components from a sleep mode to an active mode. In place of, or in combination with, pressure-triggered activation, the micro-controller 108 can activate various components according to time-triggered activation at a certain (pre-set or programmable) time, such as 2 am when levels of salivary analytes reach steady state.

(b) Oral Hygiene Device with Integrated 9-Axis Inertial Motion Sensor and Wireless Reader:

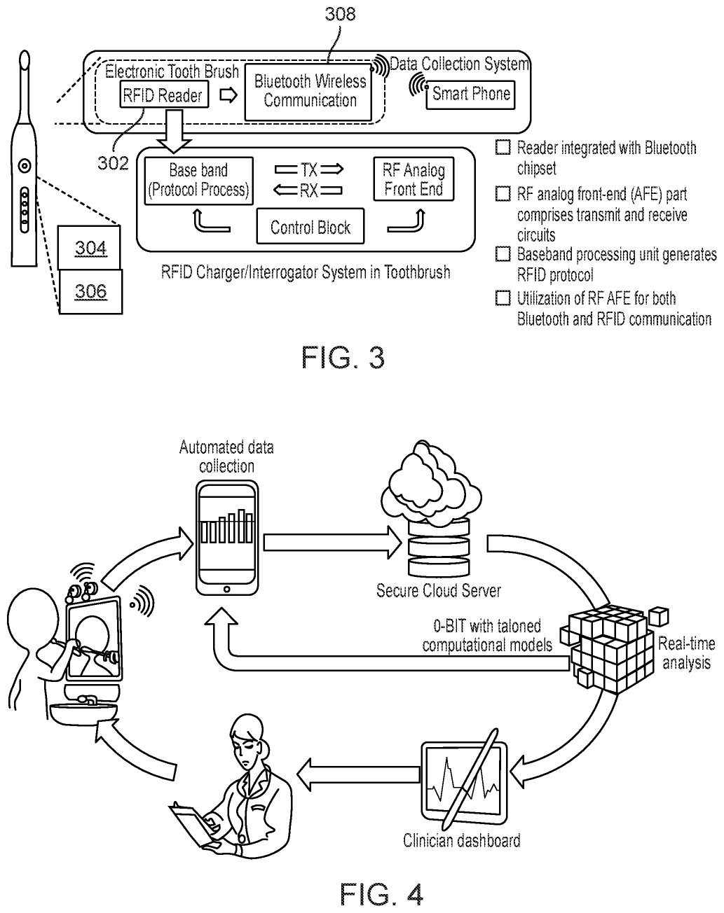

FIG. 3 shows a schematic of an oral hygiene device and its architecture and features of some embodiments. Data collected and stored by an oral appliance is retrieved by an RFID reader 302 included within a handle of an electric toothbrush (which also serves as a near-field charger to supply power to the oral appliance). In addition to the RFID reader 302, the handle of the electric toothbrush includes a multi-axis inertial motion sensor 304 (e.g., a 9-axis inertial motion sensor including a 3-axis gyroscope, a 3-axis accelerometer, and a 3-axis magnetometer) to detect physiological movements when using the toothbrush (e.g., timing, frequency, duration, pressure, and location of brushing, or hand tremors), and a micro-controller 306 to direct operation of various components of the toothbrush. The interrogated data from the oral appliance (along with any usage and other physiological movement data) are then transmitted, via a wireless communication module 308 in the form of a Bluetooth chipset (or other wireless chipset) included in the handle of the electric toothbrush, to a smartphone or mesh network, and then transmitted to a cloud server for analysis and computation of digital phenotypes or profiles (FIG. 4). Here, a ML/AI platform can translate this measured unstructured data into individual and population-level models and help explain the development of diseases, make predictions on the future development of diseases and the likely response to specific therapies and preventive measures. In place of, or in combination with, the smartphone, another portable electronic device can be used, such as a smartwatch.

(c) ML/AI Platform for Processing Toothbrush-Derived Data:

A ML/AI platform of some embodiments is implemented using computer-readable code or instructions stored in a non-transitory computer-readable storage medium. The ML/AI platform performs the following tasks:

(c1) Session-specific data collection from a heterogeneous set of sensors: The platform collects and preprocesses outputs obtained from a diverse set of sensors, including the following: (1) Inertial (e.g., electromechanical) sensors, which are located in an electric toothbrush or in another portable electronic device placed on a user (e.g., smartwatch) or in an environment of the user. These inertial sensors can, for example, record 9-axis instantaneous measurements of linear accelerations (via a 3-axis accelerometer), magnetic field (via a 3-axis magnetometer), and angular rotations (via a 3-axis gyroscope) of the toothbrush and body parts where the sensors are located. (2) Electrochemical sensors, which are located intraorally (and collect data on salivary analytes) or contained in the toothbrush (and collect data on volatile organic compounds in exhaled breath). The preprocessing stage for each type of sensor is geared to the type of data it collects and a power of a computing hardware integrated into a sensor platform. For example, outlier detection and smoothing operations of raw measurements can be executed by the sensor platform itself, or can be executed by the platform.

(c2) Privacy-aware and secure individual and population-level data storage and indexing: The data collected per individual is stored in the cloud or on dedicated servers using techniques for secure storage and compliance with the Health Insurance Portability and Accountability Act (HIPPA) standards. Temporal analysis of data and models derived from such analyses (as explained in the following stages) allow tracking of behavior and health status at the level of segment-of-one. Each individual user's data is indexed with attributes related to demographic, behavioral, and health-related conditions or status of the user. This indexing allows collective population-level data to be searched and processed based on different population segments, as desired. Thus, the data set can be parsed into overlapping segments, such as users who have Type 2 diabetes, or hypertension, or those who consume a salt-containing snack, and their various combinations. Data for each such segment of population can be analyzed to derive cohort-level models of health risk. Similarly, analysis can proceed in the inverse direction and, based on data models derived from sensor outputs, identification of additional cohorts can be made with particular risk factors. For example, detection of hand tremors (collected during the act of tooth brushing) can be used to infer stasis or progression of movement disorders (e.g., multiple sclerosis, neurodegenerative disease, stroke, and so forth). The platform can then collect data for all users with such movement anomalies and determine correlates over health conditions of the users to identify a newly-defined and medically relevant cohort. Similarly, the platform can identify temporal and range patterns in measurements of different salivary analytes and identify cohorts of users where such patterns are persistent.

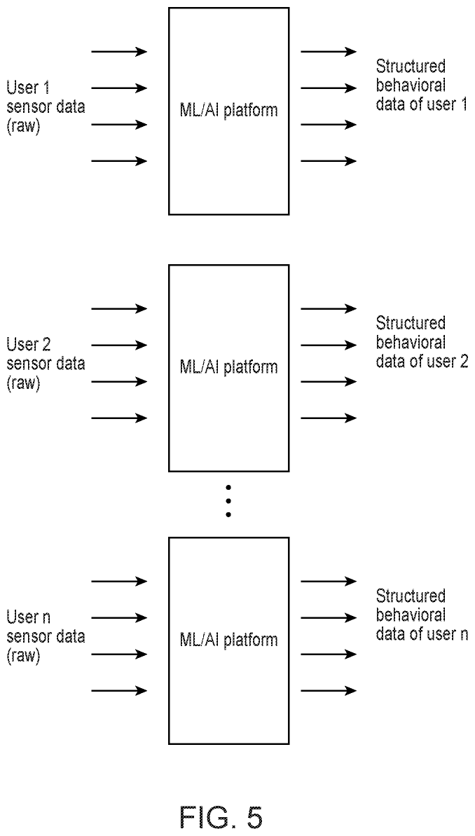

(c3) From unstructured sensor data to structured behavioral or health data—creating interpretable and multi-scale data-driven behavioral or health models: Sensor data are typically unstructured in the sense that the data represent measurements of physical quantities, such as linear accelerations, angular rotations, and magnetic fields, or measurement of salivary analytes (electrolytes or metabolites). These data sets have raw information that can be used to derive interpretable models that provide structured information for higher-level interpretation and diagnosis. The conversion of unstructured sensor data to structured behavioral or health data for individual users can be performed using Bayesian analysis in ML (see FIG. 5). Each targeted structured model or outcome has its own distribution over sensor output, and each individual has a prior distribution over the models. Posterior probabilities of models can be inverted and derived given the unstructured sensor data. An example of such a processing stage for a brushing session is mapping an output of inertial sensors to (i) a geometric three-dimensional (3-D) map of brushed regions, where dental areas are categorized into quadrants (e.g., upper left quadrant, upper right quadrant, lower left quadrant, and lower right quadrant) and into further sub-regions as desired for monitoring of brushing efficacy, (ii) a time spent brushing each region, (iii) types of micro-strokes and brushing pressure applied to each region, (iv) any extraneous but correlated movements of head or other body parts during brushing and (v) any interruptions in brushing movements. In order to achieve this mapping, the platform can use a supervised, end-to-end training approach, where sensor data are fed as input to a classifier, such as a Deep Learning (DL) network, and the classifier is trained to map such temporal sensor data sequence to different regions in a supervised manner. Such supervised approach can involve a relatively large training data set where regions brushed are tagged, for a relatively large group of individuals. This mapping also can be performed using a semi-supervised approach where physics models are used to preprocess unstructured data and the resulting physically meaningful structured information is fed to a classifier to build models to predict brushed regions. In some embodiments, this semi-supervised approach is used, since it can lead to more accurate models and involve less supervised data. Other examples of structured data include models for food and drink consumption and stress habits of individuals measured from electrochemical sensor data. For example, each type of food and drink consumed by a user can lead to different patterns of measured analytes, allowing derivation of distributions over eating habits using Bayesian Statistics. Similarly, different stress experiences can lead to different characteristic sets of analyte levels, and thus measured analyte data can be mapped to structured information about levels and types of stress being experienced by a user.

Such structured information can be then used as a data set to derive behavioral or health models of individual users as well as for grouping multiple individuals into cohorts who have similar high-level behavioral or health patterns. The automated identification of meaningful cohorts is a particularly desirable functionality of some embodiments. For example, a particular application of structured brushing behavioral data is automated labeling and recognition of individual members of a family who use the same electric toothbrush handle but different brush heads. Each individual can have a unique signature in the way one moves and operates the toothbrush and this signature is expressed in motions when brushing. Certain high-level features such as rotations of a brush head and acceleration patterns in different quadrants can be used to uniquely label and cluster brushing sessions of tens of individuals in an automated manner.

Further details and example implementations for the conversion of unstructured sensor data to structured behavioral or health data are provided below.

### Example 1: Conversion from 9-Axis Measurements to Dental Regions being Brushed

(a) A Supervised Approach:

Input: Sessions recorded with 9-axis measurements at a time instant “t” and measurements x₁(t), x₂(t), . . . , x₉(t)

Training Set:

(Input, Region i)(t)

Input: 9-axis measurements

Region i: desired output

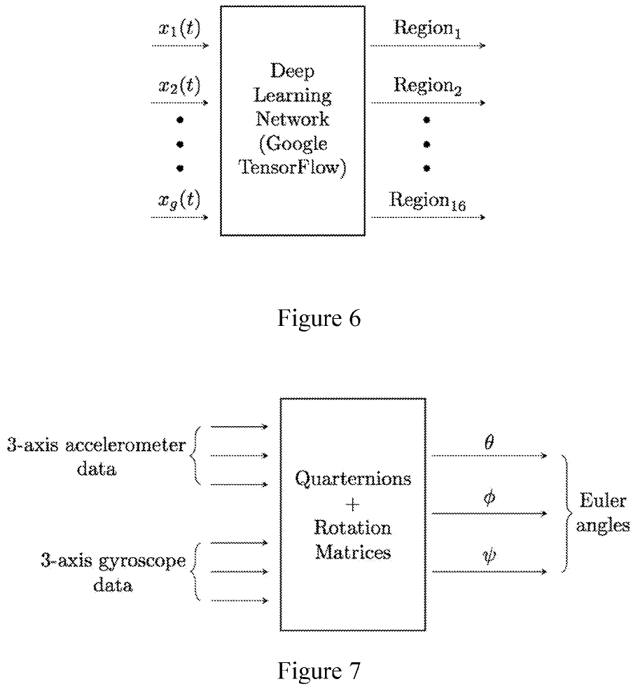

The DL network is trained to map measurements to a probability that a region being brushed is the i^{th} region. Although FIG. 6 shows a total of 16 dental regions to which mapping can be performed, more or less dental regions can be included for other implementations.

(b) A Semi-Supervised Approach Based on Physics Models:

9-axis measurements of inertial sensors in an electric toothbrush are embedded into two reference frames:

(i) Reference frame (R₁) that is attached to the inertial sensors, and

(ii) A stationary reference frame (R₂)

Since the sensors themselves move, the measurements are with respect to R₁. An orientation of the toothbrush can be represented in terms of orientation angles, namely Euler angles, with respect to the stationary reference frame.

→ Each Region i has a probability distribution over the Euler angles and angles with respect to magnetic fields (from a 3-axis magnetometer).

$$P ( \begin{matrix}θ ( t ) , φ ( t ) , ψ ( t ) , 〈 angle with magnetic north 〉 ( t ) , \\ 〈 angle with magnetic inclination direction 〉 ( t )\end{matrix} \vert Region i )$$

Notes:

1. The above probability distribution is less sensitive to inter-user and inter-session variations. Thus, this probability distribution can be derived using less data than in the case of a supervised approach.

2. Bayesian Statistics can be used to obtain

$$P ( Region i \vert \begin{matrix}\begin{matrix}θ ( t ) , φ ( t ) , ψ ( t ) , \\ accelerometer data ,\end{matrix} \\ magnetic data\end{matrix} )$$

to obtain probabilities of different dental regions at a certain time.

3. Transitions between regions can be used to render region predictions more accurate. Certain groups of regions i₁, i₂, i₃, for example, can have P(data|iᵢ)≈P(data|i₂)≈P(data|i₃), and hence, their predictions from observed data can become ambiguous.

For example, sensor data for Mandibular Right Buccal and Mandibular Left Lingual can be similar for many users. However, because their positions are different in a mouth cavity, motions performed to transition into and out of these regions are different.

Hence, any ambiguity can be resolved either by deriving models for:

a. Transitions from region j to region i₁, and from region j to region i₂, and/or

b. Transitions from region iᵢ to region j, and from region i₂ to region j.

In general, probability distributions become distinct once transitions are taken into consideration, namely P(data|i₁→j)≠P(data|i₂→j), thereby allowing for an accurate prediction of the regions i₁ and i₂ when such transitions are identified.

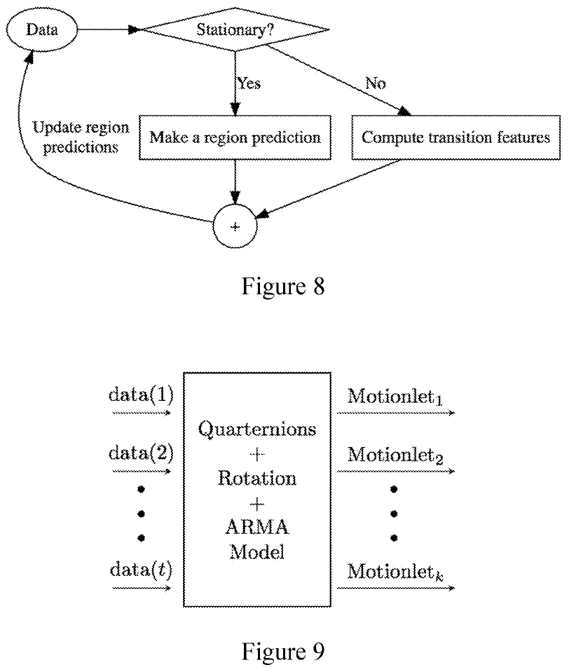

The schematics in FIG. 7 and FIG. 8 capture this processing.

### Example 2: From Unstructured Data to Motionlets or Brushing Strokes

Motionlets or brushing strokes can be specified as coordinated 3D movements that are atomic, and longer movements and activities can be constituted by a combination of such atomic motionlets. Such motionlets are performed to, for example, i) brush certain hard-to-reach regions in a mouth cavity; ii) to make transitions from one region to another region, and iii) to uniquely identify users, as each user tends to have a preferred set of motionlets or gestures when performing activities.

From a ML perspective, each motionlet is a short segment of a time-series of motion data that has a particular signature of a set of rotations and translations. For example, a motionlet can be described as a specific set of sequential rotations around x, y, z axes, and translations in the x, y, z directions. Thus a motionlet is identified by mapping a sequence of time-series sensor data to a motionlet label as shown in FIG. 9. An Auto-Regressive-Moving-Average (ARMA) model can be trained to model each motionlet i.

### Example 3: From Unstructured Data for Analytes to Disease and Health Status

Let x₁(t), x₂(t), . . . , x_{K}(t) be a time-series data representing measurements of K analytes. And let y₁(t), y₂(t), . . . , yₘ(t) be a likelihood or a degree of m different diseases or health status outcomes that are being tracked.

From an ML perspective, this can be represented for regression analysis

yᵢ(t)=ƒ_{i,θ}(x₁(t),x₂(t), . . . ,x_{K}(t)|θ)+∈ᵢ(t)

where ƒ_{i,θ}( . . . |θ) is a prediction function and θ are parameters of ƒ.

For example, in a linear regression analysis,

$${y}_{i} ( t ) = {\sum }_{j = 1}^{k} {θ}_{ij} {x}_{j} ( t ) + {ϵ}_{i} ( t )$$

The parameters θᵢⱼ are derived in a population-level digital phenotype stage, as described in the following.

Notes:

Disease or health status predictions also can be dependent on intrinsic variables or other factors particular to a user's attributes, such as age, gender, race, income level, geo-location, and other health conditions or medications.

Hence different contextual models can be derived at a population-level.

Let z₁, z₂, . . . , zₗ be factors that lead to different predictions or, in a ML perspective, these factors are referred to as the hyper-parameters, and

θᵢⱼ=gᵢⱼ(z₁,z₂, . . . ,zᵢ).

Thus prediction models are themselves conditioned on the factors that are intrinsic to the user, where zⱼ(uᵢ)=pⱼ (cumulative data of the user). These hyper-parameters can be estimated from user data, either directly from user history or inferred from user activities. For example, income-levels and food habits of a user can be estimated from web and internet activities of the user.

Similarly, daily measurements of salivary electrolytes linked to health and disease (e.g., sodium and hypertension) can be used to derive temporal snapshots of an individual's condition at a given time (patient snapshots). Then, the snapshots can be used to derive prognostic models including temporal windows allowing prediction of short, medium and long-term prognosis regarding progression to overt disease and set the stage for titrated interventions.

(c4) Deriving Normative Models for Structured Behavior Patterns: Population-Level Digital Phenotypes: The platform for user-level digital phenotype determination utilizes the following principle: An individual is characterized by how it matches and differs from population-level trends over relevant categories. Hence, in order to characterize an individual, a dictionary of categories that are relevant to a population and a distribution of variables that comprise these categories (over the population) are obtained. Thus, the platform derives structured behavioral or health patterns (which is performed in the previous stage) as well as a distribution of a population over such behavioral or health patterns before deriving individual digital phenotypes.

The previous stage provides a methodology to specify categories, and in this stage the platform determines levels or quantization of structured data so as to specify at a population-level what distributions are over the categories. For each structured variable specified in the previous stage, the platform can incrementally build a distribution. For example, the platform can (i) derive a frequency and a duration of brushing of each dental region over an entire population, (ii) condition processing on different segments of populations to obtain population segment-specific distributions, and (iii) undergo processing into finer details and condition it on different types of brush heads, different age groups, or other attributes to derive distributions mapping dependency of brushing behaviors on particular designs of brushes or on different age groups. In some embodiments, the platform can continually search over various possible combinations of structured behavioral or health variables and relevant ancillary attributes (such as age, medical conditions, geo-locations, and so forth) to derive population-level digital phenotypes. Bayesian networks and automated clustering and density estimation methodologies can be used for performing this task. Bayesian analysis can determine which variables are conditionally independent, allowing a search over combinations of variables that have greater information.

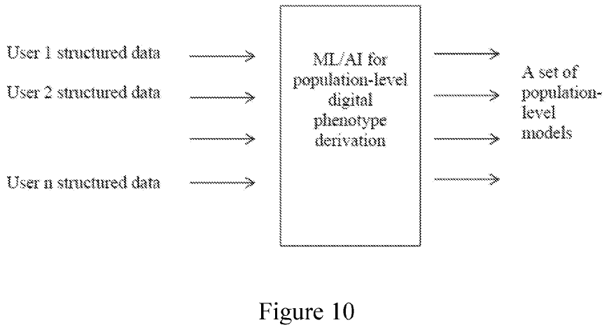

These population-level models are derived by aggregating population-level data sets composed of structured data of individual users across a population (see FIG. 10). These population-level models can lead to discoveries and allow monitoring of behavioral or health status of individual users. For example, a particular behavior pattern as a structured variable can be the amount of hand tremor that occurs during brushing sessions. This tremor can be a function of age, being more for children and less for adults and then increasing with old age. Thus, the platform can use a segmentation methodology to partition a distribution of measured levels of tremor into different age bins. For each age bin the platform can estimate the distribution and given any user the platform can determine a percentile that the user belongs for his or her age group when it comes to tremors. Thus, if someone develops tremors that are significantly above a mean, the platform can quantify a probability of such an occurrence and if the probability remains and is persistent, the platform can generate an alert for caregivers to check for progression of a neurological disease. As an example of a discovery using population-level phenotypes, the platform can identify a susceptibility of becoming stressed depending on different eating habits. Since sensors can measure data representing both levels of stress and types of food intake, the platform can identify correlations between two sets of structured variables over different population segments and determine in an automated manner population-level phenotypes where such correlations exist.

Further details and example implementations for the derivation of population-level digital phenotypes are provided below.

The ML/AI platform is used to derive an array of population-level models from population-level data sets, which are then used to derive individual user's digital phenotypes.

1. Specifying a set of attributes that are relevant to a population. These attributes can include categorical variables, such as age, gender, income level, DNA and other genetic markers, diseases, health conditions, eating habits, movement habits, lifestyle habits, and so forth.

Thus these attributes can include both attributes that are a priori considered relevant (e.g., from domain knowledge), as well as those that are identified to be relevant from population-level data sets. In the following, examples are provided on how to identify relevant attributes, and then create dictionaries, namely quantifying and specifying categories from these attributes, in an automated manner using ML/AI techniques:

### Example 1: Identifying a Target Attribute to be Relevant or not and Specifying Categories from the Attribute

A basic set of criteria can be used, such as those based on clustering and unsupervised learning in AI.

For example, consider the case of “age” as an attribute. One criterion to determine whether it is relevant can be if an observed data (sensor data) has a high variance over different age groups. If the observed data does not have high enough variance then age is likely not a relevant attribute.

Next is the question of how many different categories to be specified based on age?

Age spectrum: |1↔l₁, l₁+1↔l₂, l₂+1↔l₃, . . . , lₖ₋₁+1↔lₖ|

What should k be? Given k, what should l₁, l₂, . . . , lₖ that specify bin boundaries be?

This can be viewed as a max-information partitioning problem.

Let Pᵢ(Data|lᵢ₋₁+1≤age≤lᵢ) be a distribution of observed data given users are from the i^{th} age group. Then an optimal choice of the boundaries l₁, l₂, . . . , lₖ can be

$$( {l}_{1}^{*} , {l}_{2}^{*} , … , {l}_{k}^{*} ) = {arg max}_{{l}_{1} , {l}_{2} , … , {l}_{k}} {\sum }_{i \neq j} {D}_{KL} ( {P}_{i} , {P}_{j} )$$

That is, the optimal choice of the age-grouping boundaries maximizes a sum of Kullback-Leibler distances (KLD) between all pairs of distributions.

Thus the platform can automatically determine age categories that maximize the information content of the observed data. The optimal k (the number of age bins) is the value of k for which the distance measure achieves a maximum.

### Example 2: Creating a Dictionary of Motionlets

A dictionary of motionlets can be derived from collected data as follows.
Motion sensor data from each user is partitioned into data segments of duration T.
Each such data segment is mapped to a set of feature vectors either using a dimensionality reduction mapping such as Principal Component Analysis (PCA) or Deep Auto-encoders or using a set of physics-based features.
These feature vectors obtained from various users can then be clustered into groups using different clustering techniques such as K-Means, spectral clustering, and so forth.
Deep Generative Adversarial Networks (GANs) can be also used to model short data segments. Similarly, Recurrent Neural Networks (RNNs) can be used to compress data segments and derive clusters.
Each such cluster then represents a motionlet pattern that is relevant to the user population.
The set of the motionlets then provides a dictionary that can be used to characterize individual users.

2. Automatically Determining a Set of Cohorts in a Population.

A cohort is a joint distribution relating a set of categorical variables, namely relating a set of attributes identified in stage 1 and a set of observed data. In particular, a cohort is represented by a set of attributes (determined in the previous stage) F=(y₁, y₂, . . . , yₖ) and a set of observed sensor data D=(x₁, x₂, . . . , xₘ) (or a set of structured data derived from such observed sensor data). The cohort is then formally represented by the following probability distributions:
i. Marginal distributions: P_{F}(y₁, y₂, . . . , yₖ), P_{D}(x₁, x₂, . . . , xₘ).
ii. Conditional probability distribution of D under condition of F and conditional probability distribution of F under condition of D:

$${P}_{D \vert F} ( {x}_{1} , {x}_{2} , … , {x}_{m} \vert {y}_{1} , {y}_{2} , … , {y}_{k} ) and$$

$${P}_{F \vert D} ( {y}_{1} , {y}_{2} , … , {y}_{k} \vert {x}_{1} , {x}_{2} , … , {x}_{m} ) = \frac{\begin{matrix}{P}_{D \vert F} ( {x}_{1} , {x}_{2} , … , {x}_{m} \vert {y}_{1} , {y}_{2} , … , {y}_{k} ) \\ {P}_{F} ( {y}_{1} , {y}_{2} , … , {y}_{k} )\end{matrix}}{{P}_{D} ( {x}_{1} , {x}_{2} , … , {x}_{m} )} .$$

These probability distributions can be used to map a given user to a cohort.

Estimation of F (a set of attributes or factors), D (set of data variables) and the joint and marginal distributions can be performed by a variety of ML/AI techniques, including
i. Parametric models of distributions P such as Gaussians, mixture of Gaussians, Dirichlet, Poisson, and so forth.
ii. Non-parametric models such as Kernels, Deep Neural Networks, and so forth.

The basic operation is to identify a set of attributes that have well-defined distributions over population-level data sets.

For example a cohort can be:
y₁=Indicator variable of whether the user is Type-2 diabetic
y₂=Indicator variable of whether the user is in the age bracket: 50≤age≤70
A set of observed sensor data

$$\{ \begin{matrix}{x}_{1} \\ {x}_{2} \\ {x}_{3}\end{matrix}$$

Thus this cohort represents users that are older and have type-2 diabetes.

Then estimation is performed for

$$P ( {y}_{1} = 1 , {y}_{2} = 1 \vert measured sensor data {x}_{1} , {x}_{2} , {x}_{3} ) = P ( the user the diabetic and older \vert measured sensor data )$$

This probability distribution can be estimated using a number of supervised and unsupervised techniques, from the population-level data set.

Yet another example of a cohort could be
y₁: Indicator variable of age group
y₂: Indicator variable of presence or absence of neurological disorder such as stroke
x₁: The level of tremor while brushing

Here, P(x₁|y₁,y₂) thus represents the likelihood of tremors given the age group and whether the user has had stroke or other neurological disorders or not. If, for example, P(x₁|y₁, y₂=False) is low and x₁ is high for a user, then the user is experiencing tremors higher than normal. The reverse probability P(y₂|x₁, y₁) can be obtained to assess the likelihood of the person having a neurological disorder given the observed tremor and his or her age group.

A set of cohorts Cᵢ that constitute each population-level digital phenotype can be continually updated and additional cohorts can be identified via ML/AI search techniques.

For example, the platform can continually identify combinations of attributes or dictionary constituents and determine their related distributions and determine if these attributes have low or high variances and related information theoretic criteria such as entropy H(x) and mutual information I(x,y). The lower the uncertainty, the higher is the prediction accuracy of the attributes given the observed data.

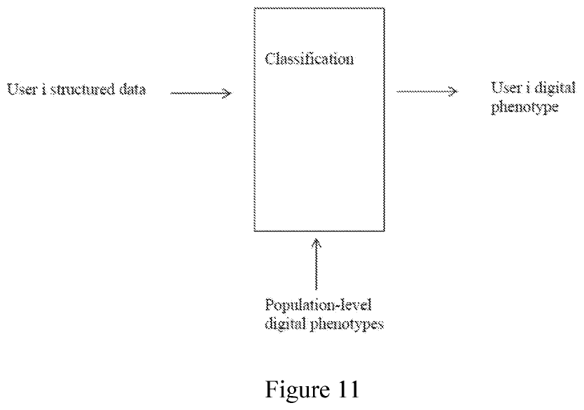

(c5) Determining Digital Phenotypes for Individuals: This stage involves deriving a vector representation of each individual user, where each coordinate of the vector representation corresponds to (i) a placement of the individual user and quantification of his or her belongingness (or a degree of affiliation or an extent of matching) in each of various population-level structured behavioral or health models and categories, (ii) demographic and other ancillary attributes that are obtained as part of the individual user's description, or (iii) any measurement pattern that is particular to the individual user and has not yet been modeled at the population-level. Thus for each user, the platform records various analyte-related categorical variables and various motion-related categorical variables (such as tremors, average brushing speed, and so forth), and derives a placement of the user in various population-level models (see FIG. 11). This vector representation is time-stamped so that the platform derives a temporal digital phenotype of each individual user.

Further details and example implementations for the derivation of individual digital phenotypes are provided below.

Once dictionaries and cohorts are determined for various population-level digital phenotypes, each individual is then mapped via a conditional probability distribution

P_{F|D}(y₁,y₂, . . . ,yₖ|x₁,x₂, . . . ,xₘ)

where y₁, y₂, . . . , yₖ are a set of attributes (e.g., presence or absence of diseases, levels of health conditions, eating habit and food intake, life style-related metrics such as level of stress, and so forth) and x₁, x₂, . . . , xₘ are a set of related observed sensor data. Similarly, each individual can be mapped via a conditional probability distribution PDIF.

The distributions P_{F|D}(y₁, y₂, . . . , yₖ|x₁, x₂, . . . , xₘ) (and P_{D|F}) are derived in stage 2 as described in the preceding section. These distributions can be represented by classifiers, or by parametric and non-parametric models.

Thus a user's digital phenotype can include granular information such as
Brushes mandibular left buccal with a pressure of 0.7 psi (in the 75% percentile of his or her age group)
Brushing efficiency (in the 75% percentile for his or her age group)
Uses motionlet #100 (a left twist of wrist 90% of the time)
   to more general information such as
Salt intake is 180% of daily recommended levels
Runs 80% chance of developing high blood pressure
Has recently reduced his or her stress levels on meditating to 50% percentile of the population

(c6) Digital Phenotypes to Health Outcomes and Personalized Diagnosis: This stage maps observed behavior patterns to actual health outcomes at the individual level. For example, a user might not be brushing his or her teeth according to a population distribution and has poor scores in his or her profile, but his or her plaque accumulation might be within norms. In this case the platform determines that brushing by the user in this way is acceptable even though the profile is indicative of daily brushing habits less than that recommended. On the other hand, an opposite situation could happen. Someone might have a propensity for faster plaque accumulation and should have extra brushing efforts. Both such situations can result in personalized feedback. The platform allows for such personalized feedback to be incorporated by creating a function that learns a mapping from the profiles to outcomes at the individual level.

(c7) Personalized Just-in-Time (JIT) Behavioral or Health Intervention: The platform is further augmented with functionality to perform JIT intervention to help users to modify behavior so as to obtain particular health outcomes. The platform incorporates a framework of Reinforcement Learning and represents the interaction between an automated intervention system and the user as a game. In particular, the platform relies on the user's digital phenotype and its mapping to an outcome. Thus, each state of the user, as determined by the digital phenotype, has an associated reward function in terms of an expected outcome. Given a particular outcome objective, an intervention is made via, for example, a reminder or a reward by recommending a change of behavior. For example, if the user forgets to take a medication and it is determined in measured salivary analytes, then a reminder is sent to take the medication for the next scheduled intake. Once such an action is taken the user receives a reward in a game that is played. If such an action is not taken, then the game does not progress. The functionality leverages the derivation of detailed and accurate digital phenotypes and their correlation with outcomes. A digital phenotype should accurately reflect an actual and current state of a user. The game and intervention functionality can be implemented as an overlay service on top of a basic framework to guide the user and personalize the intervention strategies to reach a particular outcome.

(c8) Beyond Dental Outcomes—Mouth as a Portal for Health Biometrics: As explained above, a set of sensors extend beyond those for dental outcomes, and encompass sensors that measure a range of data on health-related analytes and motion-related behavior. The platform can use large-scale data to automatically discover patterns in the measured data. These patterns can then be correlated with disease risk stratification, and allow remote monitoring of health, diet patterns, and individualized interventions.

Examples of applications of the platform of some embodiments include:

(1) The data-driven models can correlate with various health outcomes, allowing insurance agencies to assign risk likelihood to individual patients.

(2) The data sets can be suited for large epidemiological studies to determine effects of drugs, food policies and public health policies. Different habits, food sources, and health policies can be manifested as patterns and cohorts that are most impacted in the data sets and models.

(3) A software application can be developed that obtains food intake patterns based on measured analytes. A user can subscribe to a service that provides daily summaries of food intake ingredients and estimated calories. The service can also provide an automated feedback strategy. Digital phenotypes can be used to customize intervention strategies. A similar service can be implemented for the detection of neuromuscular diseases or assessing brushing habits in at-risk individuals.

(4) By turning toothbrushes into smart, connected ones, manufacturers can leverage the platform to establish improved customer engagement, and provide personalized services and experiences. Manufacturers can leverage the platform to provide additional functionality or track performance and usage by consumers. For example, brushing behavior can be used to monitor inventory levels or manage maintenance and repair. Manufacturers can perform track-and-trace to identify a physical location of products, measure environmental factors such as temperature and humidity to ensure operating efficiency or predict failures, monitor actual usage for compliance with warranty terms or contractual agreements and effectively replenish brush heads in a personalized and timely manner instead of generic monthly subscriptions. Digital phenotypes can provide a deeper level of customer engagement and turn a static relationship that ends with a sale into an ongoing relationship with a consumer. Examples of such engagement can include:
Personalization of toothbrushes for consumers using digital phenotypes: Digital phenotypes of past and current users can be used to predict design and functionalities that can best serve a growing cohort. Initially, a digital phenotype of a new customer can include partial information based on attributes that are shared by the new consumer, such as age, weight, height, gender, health conditions if any, and eating habits. It can also include more detailed information, such as 3D scans and models of the consumer's grip and hand, as well as 3D scans of the teeth and oral cavity. Based on population-level digital phenotypes, such information can be used to determine digital doppelgangers or avatars of the consumer, which in turn can guide the design of a toothbrush itself. Examples of design parameters that such personalization can concern are: (a) physical design and usability considerations, such as grip measurements of a brush handle, and specific design of a brush head to match the dentition and oral cavity of the consumer—this can avoid mechanical failures and also inefficiency in brushing outcome; and also (b) bio-sensing design considerations, such as a set of sensors (e.g., breath analysis sensors) to be included in the toothbrush so as to provide relevant information about the consumer.
Continued personalization of experience and engagement: As a consumer continues to use a connected toothbrush and interact with the platform, his or her digital phenotype will include information of greater granularity. As each such additional information is included, it can be used to provide additional services, such as alerts and analytics on the status of his or her oral health, and also of particular health conditions that a personalized set of sensors are targeted to monitor. In addition, his or her digital phenotype can be used to guide and select intervention strategies that can help engage and guide the consumer to achieve particular goals, whether it concerns oral or general health.

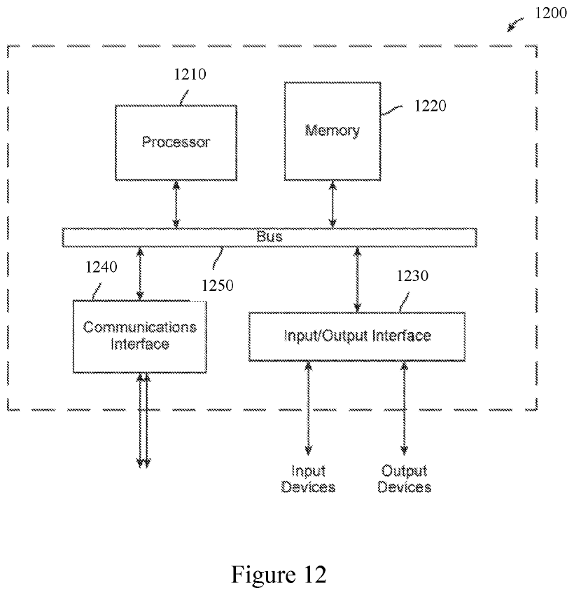

FIG. 12 shows an example of computing device 1200 that includes a processor 1210, a memory 1220, an input/output interface 1230, and a communications interface 1240. A bus 1250 provides a communication path between two or more of the components of computing device 1200. The components shown are provided by way of example and are not limiting. Computing device 1200 may have additional or fewer components, or multiple of the same component.

Processor 1210 represents one or more of a microprocessor, microcontroller, an application-specific integrated circuit (ASIC), and a field-programmable gate array (FPGA), along with associated logic.

Memory 1220 represents one or both of volatile and non-volatile memory for storing information. Examples of memory include semiconductor memory devices such as erasable programmable read-only memory (EPROM), electrically erasable programmable read-only memory (EEPROM), random-access memory (RAM), and flash memory devices, discs such as internal hard drives, removable hard drives, magneto-optical, compact disc (CD), digital versatile disc (DVD), and Blu-ray discs, memory sticks, and the like. The functionality of the ML/AI platform of some embodiments can be implemented as computer-readable instructions in memory 1220 of computing device 1200, executed by processor 1210.

Input/output interface 1230 represents electrical components and optional instructions that together provide an interface from the internal components of computing device 1200 to external components. Examples include a driver integrated circuit with associated programming.

Communications interface 1240 represents electrical components and optional instructions that together provide an interface from the internal components of computing device 1200 to external networks.

Bus 1250 represents one or more connections between components within computing device 1200. For example, bus 1250 may include a dedicated connection between processor 1210 and memory 1220 as well as a shared connection between processor 1210 and multiple other components of computing device 1200.

Some embodiments of this disclosure relate to a non-transitory computer-readable storage medium having computer-readable code or instructions thereon for performing various computer-implemented operations. The term “computer-readable storage medium” is used to include any medium that is capable of storing or encoding a sequence of instructions or computer code for performing the operations, methodologies, and techniques described herein. The media and computer code may be those specially designed and constructed for the purposes of the embodiments of the disclosure, or they may be of the kind available to those having skill in the computer software arts. Examples of computer-readable storage media include those specified above in connection with memory 1220, among others.

Examples of computer code include machine code, such as produced by a compiler, and files containing higher-level code that are executed by a processor using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computing device) to a requesting computer (e.g., a client computing device or a different server computing device) via a transmission channel. Another embodiment of the disclosure may be implemented in hardwired circuitry in place of, or in combination with, processor-executable software instructions.

### EXAMPLE EMBODIMENTS

In some embodiments, an oral appliance includes: (1) a salivary sensor module including multiple sensors responsive to levels of different salivary analytes, and configured to generate output signals corresponding to the levels of the different salivary analytes; (2) a wireless communication module; and (3) a micro-controller connected to the salivary sensor module and the wireless communication module, and configured to derive the levels of the different salivary analytes from the output signals and direct the wireless communication module to convey the levels of the different salivary analytes to an external device.

In some embodiments of the oral appliance, the salivary sensor module includes a readout circuit connected to the multiple sensors and configured to generate the output signals.

In some embodiments of the oral appliance, the readout circuit is configured to sequentially obtain measurements across the multiple sensors.

In some embodiments of the oral appliance, the oral appliance further includes a temperature sensor configured to generate a calibration signal responsive to a local temperature, and wherein the readout circuit is configured to adjust the measurements according to the calibration signal.

In some embodiments of the oral appliance, the micro-controller is configured to activate the salivary sensor module according to time-triggered activation.

In some embodiments of the oral appliance, the oral appliance further includes a pressure sensor configured to generate an event-triggered signal, and wherein the micro-controller is connected to the pressure sensor and is configured to activate the salivary sensor module in response to the event-triggered signal.

In some embodiments of the oral appliance, the wireless communication module includes a Radio Frequency Identification (RFID) tag.

In additional embodiments, a monitoring system includes: (1) the oral appliance of any of the foregoing embodiments; and (2) an oral hygiene device including a wireless reader configured to retrieve the levels of the different salivary analytes from the oral appliance.

In some embodiments of the monitoring system, the wireless reader is configured to supply power to the oral appliance through the wireless communication module of the oral appliance.

In some embodiments of the monitoring system, the wireless reader includes an RFID reader.

In some embodiments of the monitoring system, the oral hygiene device is configured as an electric toothbrush.

In some embodiments of the monitoring system, the oral hygiene device includes a multi-axis inertial sensor.

In further embodiments, a computer-implemented method includes: (1) deriving structured data of a user from sensor data collected for the user; (2) collecting attributes of the user; (3) aggregating the structured data of the user and the attributes of the user with structured data of additional users and attributes of the additional users to obtain a population-level data set; (4) identifying a set of cohorts from the population-level data set; and (5) deriving a profile of the user indicative of an extent of matching of the user with the set of cohorts.

In some embodiments of the computer-implemented method, the method further includes generating a feedback to the user according to the profile of the user.

In some embodiments of the computer-implemented method, the sensor data include data on salivary analytes of the user, and deriving the structured data of the user includes identifying a food or drink intake of the user from the data on the salivary analytes.

In some embodiments of the computer-implemented method, the sensor data include data on salivary analytes of the user, and deriving the structured data of the user includes identifying a health or stress condition of the user from the data on the salivary analytes.

In some embodiments of the computer-implemented method, the sensor data include inertial sensor data of a toothbrush operated by the user, and deriving the structured data of the user includes identifying dental regions brushed by the user from the inertial sensor data.

In some embodiments of the computer-implemented method, the sensor data include inertial sensor data of a toothbrush operated by the user, and deriving the structured data of the user includes identifying a set of motionlets from the inertial sensor data.

In some embodiments of the computer-implemented method, the attributes of the user include attributes related to at least one of demographic, behavioral, or health condition of the user.

In some embodiments of the computer-implemented method, identifying the set of cohorts includes deriving a conditional probability distribution for each of the set of cohorts.

In some embodiments of the computer-implemented method, deriving the profile of the user includes identifying a placement of the user relative to the conditional probability distribution.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects.

As used herein, the terms “connect,” “connected,” and “connection” refer to an operational coupling or linking. Connected objects can be directly coupled to one another or can be indirectly coupled to one another, such as via another set of objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

1. An oral appliance comprising:
a salivary sensor module including multiple sensors responsive to levels of different salivary analytes, and configured to generate output signals corresponding to the levels of the different salivary analytes;
a wireless communication module; and
a micro-controller connected to the salivary sensor module and the wireless communication module, and configured to derive the levels of the different salivary analytes from the output signals and direct the wireless communication module to convey the levels of the different salivary analytes to an external device.

2. The oral appliance of claim 1, wherein the salivary sensor module includes a readout circuit connected to the multiple sensors and configured to generate the output signals.

3. The oral appliance of claim 2, wherein the readout circuit is configured to sequentially obtain measurements across the multiple sensors.

4. The oral appliance of claim 2, further comprising a temperature sensor configured to generate a calibration signal responsive to a local temperature, and wherein the readout circuit is configured to adjust the measurements according to the calibration signal.

5. The oral appliance of claim 1, wherein the micro-controller is configured to activate the salivary sensor module according to time-triggered activation.

6. The oral appliance of claim 1, further comprising a pressure sensor configured to generate an event-triggered signal, and wherein the micro-controller is connected to the pressure sensor and is configured to activate the salivary sensor module in response to the event-triggered signal.

7. The oral appliance of claim 1, wherein the wireless communication module includes a Radio Frequency Identification (RFID) tag.

8. A monitoring system comprising:
the oral appliance of claim 1; and
an oral hygiene device including a wireless reader configured to retrieve the levels of the different salivary analytes from the oral appliance.

9. The monitoring system of claim 8, wherein the wireless reader is configured to supply power to the oral appliance through the wireless communication module of the oral appliance.

10. The monitoring system of claim 8, wherein the wireless reader includes an RFID reader.

11. The monitoring system of claim 8, wherein the oral hygiene device is configured as an electric toothbrush.

12. The monitoring system of claim 8, wherein the oral hygiene device includes a multi-axis inertial sensor.

13. A computer-implemented method comprising:
deriving structured data of a user from sensor data collected for the user;
collecting attributes of the user;
aggregating the structured data of the user and the attributes of the user with structured data of additional users and attributes of the additional users to obtain a population-level data set;
identifying a set of cohorts from the population-level data set; and
deriving a profile of the user indicative of an extent of matching of the user with the set of cohorts.

14. The computer-implemented method of claim 13, further comprising generating a feedback to the user according to the profile of the user.

15. The computer-implemented method of claim 13, wherein the sensor data include data on salivary analytes of the user, and deriving the structured data of the user includes identifying a food or drink intake of the user from the data on the salivary analytes.

16. The computer-implemented method of claim 13, wherein the sensor data include data on salivary analytes of the user, and deriving the structured data of the user includes identifying a health or stress condition of the user from the data on the salivary analytes.

17. The computer-implemented method of claim 13, wherein the sensor data include inertial sensor data of a toothbrush operated by the user, and deriving the structured data of the user includes identifying dental regions brushed by the user from the inertial sensor data.

18. The computer-implemented method of claim 13, wherein the sensor data include inertial sensor data of a toothbrush operated by the user, and deriving the structured data of the user includes identifying a set of motionlets from the inertial sensor data.

19. The computer-implemented method of claim 13, wherein the attributes of the user include attributes related to at least one of demographic, behavioral, or health condition of the user.

20. The computer-implemented method of claim 13, wherein identifying the set of cohorts includes deriving a conditional probability distribution for each of the set of cohorts.

21. The computer-implemented method of claim 20, wherein deriving the profile of the user includes identifying a placement of the user relative to the conditional probability distribution.
